(19) United States
(12) Patent Application Publication
(10) Pub. No.: US 2023/0331780 A1
(43) Pub. Date: Oct. 19, 2023

(54) COMPOSITIONS AND METHODS FOR THE TREATMENT OF ALZHEIMER'S DISEASE

(71) Applicant: The Trustees of Columbia University in the City of New York, New York, NY (US)

(72) Inventors: Elizabeth Bradshaw, Hastings on Hudson, NY (US); Wassim Elyaman, Hastings on Hudson, NY (US)

(21) Appl. No.: 18/104,105

(22) Filed: Jan. 31, 2023

Related U.S. Application Data
(63) Continuation of application No. PCT/US2021/043679, filed on Jul. 29, 2021.
(60) Provisional application No. 63/059,881, filed on Jul. 31, 2020.

(51) Int. Cl. C07K 7/08; A61K 45/06; A61P 25/28; A61K 47/54

(52) U.S. Cl. CPC C07K 7/08; A61K 45/06; A61P 25/28; A61K 47/54

(57) ABSTRACT

The present disclosure provides compositions and methods for disrupting the interaction of a siglec protein and its binding partners, and thereby treating neurodegenerative disease.

### CROSS-REFERENCE TO RELATED APPLICATIONS

This application is a Continuation of International Patent Application PCT/US2021/043679 filed 29 Jul. 2021; which claims priority to U.S. Provisional Patent Application 63/059,881 filed 31 Jul. 2020; each of which is incorporated herein by reference for all purposes.

### STATEMENT REGARDING FEDERALLY SPONSORED RESEARCH OR DEVELOPMENT

This invention was made with government support under AG043617 awarded by the National Institutes of Health. The government has certain rights in the invention.

### REFERENCE TO AN ELECTRONIC SEQUENCE LISTING

The contents of the electronic sequence listing (entitled Columbia480US-CON.xml; Size: 25 KB; created 31 Jan. 2023) is herein incorporated by reference in its entirety.

### BACKGROUND

Alzheimer's disease (AD) is a progressive mental deterioration and form of dementia that often occurs in old age due to generalized degeneration of the brain. AD is a neurodegenerative disorder that affects memory and other cognitive functions, and is the most common cause of dementia. An Alzheimer's Disease Association (ADA) survey shows that 5.4 million people in the United States (US) currently have AD and 13.5 million are expected to have AD within the next 40 years. AD affects over 26 million people worldwide and currently there is no cure for the disease. With the growing number of people living to older ages, there is an urgency to find methods and compositions useful in the treatment and prevention of AD.

### SUMMARY

In certain aspects, provided herein are peptides that disrupt the interaction between a Siglec protein (e.g., CD33) and a ligand of the Siglec protein (e.g., CD45), as well as pharmaceutical compositions comprising such peptides, methods of using such peptides, nucleic acids encoding such peptides, and cells expressing such peptides. As disclosed herein, peptides comprising the amino acid of SEQ ID NO. 1 exhibit increased CD45 phosphatase activity, decreased amounts of Amyloid-β in cells, and increased neuronal survival as compared to peptides comprising the amino acid of SEQ ID NO. 2.

In certain embodiments, provided herein are peptides that comprise an amino acid sequence having at least 83% (e.g., 83%, 84%, 85%, 86%, 87%, 88%, 89%, 90%, 91%, 92%, 93%, 94%, 95%, 96%, 97%, 98%, or 99%) sequence identity to the amino acid sequence of SEQ ID NO: 1 or SEQ ID NO: 3-20, wherein the peptide comprises an arginine, lysine, or phenylalanine residue at the position corresponding to the first or second amino acid of SEQ ID NO: 1 or SEQ ID NO: 3-20. In some embodiments, the peptide comprises an amino acid sequence identical to the amino acid sequence of SEQ ID NO: 1 or SEQ ID NO: 3-20.

In some embodiments, the peptide is at least 11 amino acids (e.g., at least 11 amino acids, at least 12 amino acids, at least 13 amino acids, etc.) in length. In some embodiments, the peptide is no more than 30 amino acids (e.g., no more than 28 amino acids, no more than 25 amino acids, no more than 22 amino acids, no more than 18 amino acids, no more than 15 amino acids, no more than 12 amino acids, etc.) in length.

In some embodiments, the peptide comprises a chemical modification. For example, the peptide is chemically modified with polyethylene glycol (PEG), a glycan, an acetic acid, an amide, a fatty acid, a phosphoryl group, a methyl group, or a combination thereof; small residues Pro, Ala and Ser (PAS); polyglycerol, polyoxazoline, polyamino acid, polyacylamide, polyvinylpyrrolidone, zwitterionic polymer, biopolymer, dendrimer, polyether, or polyethylene glycol, or a combination thereof; cholesterol, cholestene, cholestane, cholestadiene, oxysterol, or a combination thereof; or palmitate, myristolate, albumin, or a combination thereof;

In some embodiments, the peptide comprises a sialic-acid binding domain.

In some embodiments, the amino acid at the N-terminus of the peptide is arginine, lysine, or phenylalanine.

In some embodiments, the peptide inhibits an interaction between a Siglec protein (e.g., CD33) and a ligand of the Siglec protein (e.g., CD45).

In certain embodiments, the peptides disclosed herein compete with a peptide of SEQ ID NO: 1 or SEQ ID NO: 3-20 for binding to a ligand of a Siglec protein (e.g. CD33).

In certain embodiments, provided herein is a pharmaceutical composition comprising a peptide described herein. In some embodiments, the pharmaceutical composition further comprises a pharmaceutically acceptable excipient. In some embodiments, the pharmaceutical composition is formulated for extended release.

In certain embodiments, provided herein is a method of inhibiting an interaction between a Siglec protein (e.g., CD33) and a ligand of the Siglec protein (e.g., CD45) on a cell surface (e.g., myeloid cell surface), comprising contacting the ligand of the Siglec protein with a peptide described herein. In some embodiments, inhibiting the interaction between the Siglec protein (e.g., CD33) and the ligand of the Siglec protein (e.g., CD45) decreases an amount of an Amyloid-β peptide (e.g., Amyloid-β peptide 1-42) in the cell (e.g., myeloid cell). In some embodiments, inhibiting the interaction between the Siglec protein (e.g., CD33) and CD45 increases CD45 phosphatase activity.

In certain embodiments, provided herein is a method of treating or preventing a neurodegenerative disease (e.g., Alzheimer's disease (AD)) in a subject comprising administering to the subject a therapeutically effective amount of a pharmaceutical composition described herein. In some embodiments, the administration is subcutaneous injection, intravenous injection, intramuscular injection, intrathecal injection, microneedle injection, intravenous infusion, oral administration, sublingual administration, or intranasal administration.

In some embodiments, the administration decreases the amount of Amyloid-β peptide (e.g., Amyloid-β peptide 1-42) in the nervous system of the subject. In some embodiments, the Amyloid-β peptide (e.g., Amyloid-β peptide 1-42) is a parenchymal Amyloid-β peptide. In some embodiments, the Amyloid-β peptide (e.g., Amyloid-β peptide 1-42) is exogenous Amyloid-β peptide. In some embodiments, the Amyloid-β peptide (e.g., Amyloid-β peptide 1-42) is endogenous Amyloid-β peptide. In some embodiments, the Amyloid-β peptide is a pathogenic Amyloid-β peptide (e.g., Amyloid-β peptide 1-42).

In some embodiments, the method further comprises further comprising conjointly administering an additional therapeutic (e.g., a cholinesterase inhibitor, memantine, donepezil, rivastigmine, suvorextant, or aducanumab) to the subject.

In certain embodiments, provided herein is a method of making an inhibitor of an interaction between a Siglec protein and a ligand of the Siglec protein, comprising synthesizing a peptide described herein.

In certain embodiments, provided herein is a nucleic acid encoding the peptide described herein.

In certain embodiments, provided herein is a vector comprising the nucleic acid described herein.

In certain embodiments, provided herein is a cell comprising the vector described herein.

In some embodiments, provided herein is a composition comprising a peptide having at least 80% sequence identity to the amino acid sequence of SEQ ID NO:1 or SEQ ID NO: 3-20. In certain embodiments the peptide has the amino acid sequence of SEQ ID NO:1 or SEQ ID NO: 3-20. In some embodiments the composition further comprises a pharmaceutically acceptable excipient. In other embodiments the composition is formulated for extended release.

In some embodiments, provided herein is a method of disrupting an interaction of a CD33 protein and a protein that binds to CD33, comprising contacting the CD33 protein with a composition comprising a peptide having at least 80% sequence identity to the amino acid sequence of SEQ ID NO:1 or SEQ ID NO: 3-20. In certain embodiments the peptide has the amino acid sequence of SEQ ID NO:1 or SEQ ID NO: 3-20. In certain embodiments the protein that binds to CD33 is a CD45 protein. In some embodiments the CD33 and CD45 proteins are located in vivo. In further embodiments the CD33 and CD45 proteins are located in the brain of a subject. In yet further embodiments the CD33 and CD45 proteins are located in the brain of a human subject.

In some embodiments, provided herein is a method of disrupting an interaction of CD33 and CD45 proteins, comprising contacting the CD33 or CD45 protein with a composition comprising a peptide having at least 80% sequence identity to the amino acid sequence of SEQ ID NO:1 or SEQ ID NO: 3-20. In certain embodiments the peptide has the amino acid sequence of SEQ ID NO:1 or SEQ ID NO: 3-20. In particular embodiments the CD33 and CD45 proteins are located in vivo. In some embodiments the CD33 and CD45 proteins are located in the brain of a subject. In other embodiments the CD33 and CD45 proteins are located in the brain of a human subject.

In some embodiments, provided herein is a method of treating Alzheimer's disease in a patient, comprising administering to the patient a therapeutically effective amount of a composition comprising a peptide having at least 80% sequence identity to the amino acid sequence of SEQ ID NO:1 or SEQ ID NO: 3-20. In certain embodiments the peptide has the amino acid sequence of SEQ ID NO:1 or SEQ ID NO: 3-20. In some embodiments the composition further comprises a pharmaceutically acceptable excipient. In other embodiments the composition is formulated for extended release. In additional embodiments the method further comprises administering to the patient a cholinesterase inhibitor, memantine, donepezil, rivastigmine, suvorextant, or aducanumab.

### DESCRIPTION OF THE DRAWINGS

Those of skill in the art will understand that the drawings, described below, are for illustrative purposes only. The drawings are not intended to limit the scope of the present teachings in any way.

FIG. 1 shows disruption of sialic acid-dependent CD33-CD45 protein-protein interaction using a peptide that mimics the sialic acid-binding domain of Siglecs (SEQ ID NO: 1; Peptide #1) in comparison to a control peptide (SEQ ID NO: 2; Peptide #4).

FIG. 2 shows disruption of sialic acid-dependent CD33-CD45 protein-protein interaction with SEQ ID NO: 1 (peptide #1) increases CD45 phosphatase activity in comparison to control peptide SEQ ID NO: 2 (peptide #4) and no peptide.

FIG. 3A and FIG. 3B show administration of SEQ ID NO: 1 (peptide #1) increased clearance of exogenous amyloid in the mouse brain in comparison to control peptide SEQ ID NO: 2 (peptide #4) and control vehicle. A representative image from an animal in each of the three groups is shown (FIG. 3A). Quantification of Amyloid-beta 1-42 in the hippocampus from the three mouse groups is shown (FIG. 3B).

FIG. 4 shows peripheral delivery and intraventricular delivery of SEQ ID NO: 1 increased amyloid clearance in the mouse brain. Quantification of Amyloid-beta 1-42 in the parenchyma from each mouse group are shown with a marked decrease of Amyloid-beta 1-42 in mice that received SEQ ID NO: 1 (Peptide #1) compared to those that did not receive peptide.

FIG. 5A and FIG. 5B shows administration of SEQ ID NO: 1 (peptide #1) increased clearance of endogenous amyloid in the brains of 5×FAD transgenic mice in comparison to control vehicle. A representative image from an animal in each of the three groups is shown (FIG. 5A). Quantification of Amyloid-beta 1-42 in the hippocampus from the three mouse groups is shown (FIG. 3B).

FIG. 6 shows administration of SEQ ID NO: 1 (peptide #1) increased neuronal survival in 5×FAD transgenic mice administered exogenous amyloid-beta 1-42 in comparison to 5×FAD animals given the same exogenous amyloid-beta 1-42 but treated with a control peptide (Scrambled) or vehicle. Quantification of signal associated with NeuN positive neurons in Layer 5 of the piriform cortex is shown (FIG. 3B).

### DETAILED DESCRIPTION

The present invention is based, at least in part, on the discovery of peptides that disrupt the interaction between a Siglec protein (e.g., CD33) and a ligand of a Siglec protein (e.g., CD45 (e.g., CD45 expressed in myeloid cells)). A study showed that targeting this protein-protein interaction using a small peptide has protective effects in reducing the amount of toxic Amyloid-β peptide (e.g., Amyloid-β peptide 1-42). These results have significant implications in providing a treatment or prevention of neurodegenerative diseases (e.g., Alzheimer's disease (AD)).

Accordingly, the present invention relates, in part, to peptides that disrupt the interaction between a Siglec protein (e.g., CD33) and a ligand of the Siglec protein (e.g., CD45), as well as pharmaceutical compositions comprising such peptides, methods of using such peptides, nucleic acids encoding such peptides, and cells expressing such peptides.

In some embodiments, described herein are peptides that disrupt the interaction between CD33 and CD45 proteins. The disclosed peptides can have at least about 80% sequence identity to SEQ ID NO: 1 or SEQ ID NO: 3-20, and substantially retaining CD33-CD45 disrupting activity or increased stability. In some embodiments, the disclosed peptides can have at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity to SEQ ID NO: 1 or SEQ ID NO: 3-20, and substantially retaining CD33-CD45 disrupting activity or increased stability.

### Definitions

The following definitions and methods are provided to better define the present invention and to guide those of ordinary skill in the art in the practice of the present invention. Unless otherwise noted, terms are to be understood according to conventional usage by those of ordinary skill in the relevant art.

As used herein, a “neurodegenerative disease” refers to a disease that results in progressive loss of structure or function of neurons, including death of neurons, such as, but not limited to, Alzheimer's disease. Such a disease may exhibit a number of symptoms, including, but not limited to, seizures, loss of motor and/or cognitive function, and the like. A neurodegenerative disease in accordance with the invention may encompass a CNS degenerative disease, or other diseases producing similar symptoms or having a common etiology. In some cases, orthologous genes in different species may exhibit similar mutations, thus resulting in similar diseases between species. For this reason, some species may serve as useful models for disease in other species.

The phrase “treating a neurodegenerative disease” refers to ameliorating the effects of, or delaying, halting or reversing the progress of, or delaying or preventing the onset of, or slowing the progression of a neurodegenerative disease as defined herein.

As used herein, a “therapeutic compound” refers to a molecule, such as a protein, peptide, polypeptide, a carbohydrate, an antibody or antibody fragment, a small molecule, or the like, that is not functionally present in cells of the subject or the like, and/or that will have a therapeutic benefit when delivered to cells of the subject.

As used herein, “subject” or “patient” refers to animals, including mammals, who are treated with the pharmaceutical compositions or in accordance with the methods described herein.

As used herein, “pharmaceutically acceptable carrier,” “carrier,” “medium,” or “biologically compatible carrier” refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio.

As used herein, “Siglec protein” or “Sialic acid-binding immunoglobulin-type lectin protein” are cell surface proteins that bind sialic acid. An exemplary Siglec protein is CD33. A Siglec protein may bind to a ligand of the Siglec protein (e.g., CD45).

“Identity” as between nucleic acid sequences of two nucleic acid molecules can be determined as a percentage of identity using known computer algorithms such as the “FASTA” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)).

The term “subject” refers to any healthy animal, mammal or human, or any animal, mammal or human afflicted with a neurodegenerative degenerative disease, e.g., Alzheimer's disease (AD). The term “subject” is interchangeable with “patient.”

As used herein, a therapeutic that “prevents” a disorder or condition refers to a that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to a subject of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

As used herein, the phrase “conjoint administration” refers to any form of administration of two or more different therapeutic compositions such that the second composition is administered while the previously administered therapeutic composition is still effective in the body (e.g., the two compositions are simultaneously effective in the patient, which may include synergistic effects of the two compositions). For example, the different therapeutic compositions can be administered either in the same formulation or in a separate formulation, either concomitantly or sequentially. In certain embodiments, the different therapeutic compositions can be administered within one hour, 12 hours, 24 hours, 36 hours, 48 hours, 72 hours, or a week of one another. Thus, an individual who receives such treatment can benefit from a combined effect of different therapeutic compositions.

The term “polypeptide” refers to a polymer of amino acids and its equivalent and does not refer to a specific length of the product; thus, “peptides”, “oligopeptides” and “proteins” are included within the definition of a polypeptide. This term also does not refer to, or exclude modifications of the polypeptide, for example, glycosylations, acetylations, phosphorylations, etc. Included within the definition are, for example, polypeptides containing one or more analogs of an amino acid (including, for example, natural amino acids, etc.), polypeptides with substituted linkages as well as other modifications known in the art, both naturally and non-naturally occurring.

As used herein, the term “nucleic acid” is used in its broadest sense and encompasses any compound and/or substance that includes a polymer of nucleotides of any length, either deoxyribonucleotides or ribonucleotides, or analogs thereof. These polymers are often referred to as polynucleotides. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, synthetic polynucleotides, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component.

“Vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops, which, in their vector form are not bound to the chromosome. In the present specification, “plasmid” and “vector” are used interchangeably as the plasmid is the most commonly used form of vector. However, as will be appreciated by those skilled in the art, the invention is intended to include such other forms of expression vectors which serve equivalent functions and which become subsequently known in the art.

A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription.

A “transcribable nucleic acid molecule” as used herein refers to any nucleic acid molecule capable of being transcribed into a RNA molecule. Methods are known for introducing constructs into a cell in such a manner that the transcribable nucleic acid molecule is transcribed into a functional mRNA molecule that is translated and therefore expressed as a protein or peptide product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see, e.g., Sambrook and Russel, Condensed Protocols from Molecular Cloning: A Laboratory Manual, Cold Spring Harbor Laboratory Press, 2006, ISBN-10: 0879697717; Ausubel et al., Short Protocols in Molecular Biology, 5th ed., Current Protocols, 2002, ISBN-10: 0471250929; Sambrook and Russel, Molecular Cloning: A Laboratory Manual, 3d ed., Cold Spring Harbor Laboratory Press, 2001, ISBN-10: 0879695773; Elhai and Wolk, 1988. Meth. Enzymol. 167:747-754, 1988); Studier, Protein Expr. Purif. 41:207-234, 2005; Gellissen, ed., Production of Recombinant Proteins: Novel Microbial and Eukaryotic Expression Systems, Wiley-VCH, 2005, ISBN-10: 3527310363; Baneyx, Protein Expression Technologies, Taylor & Francis, 2004, ISBN-10: 0954523253).

The “transcription start site” or “initiation site” is the position surrounding the first nucleotide that is part of the transcribed sequence, which is also defined as position+1. With respect to this site all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

“Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism such as a bacterium, cyanobacterium, animal or a plant into which a heterologous nucleic acid molecule has been introduced. The nucleic acid molecule can be stably integrated into the genome as generally known in the art. Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

“Wild-type” refers to a virus or organism found in nature without any known mutation.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Maryland, 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutical active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

As used herein, “introducing,” “delivering,” and “administering” refer to the therapeutic introduction of a therapeutic compound to a subject. Administration may take place by any route.

As used herein, “regenerative” refers to the ability of a substance to restore, supplement, or otherwise rehabilitate the natural function of a tissue. This ability may be conferred by, for example, treating a dysfunctional tissue with a regenerative therapeutic compound. Regenerative compounds treat dysfunctional tissue by helping to restore the natural activity of dysfunctional tissue.

The terms “restore,” “restoration” and “correct” are used interchangeably herein and refer to the regrowth, augmentation, supplementation, and/or replacement of a defective tissue with a new and preferentially functional tissue. The terms include the complete and partial restoration of a defective tissue. Defective tissue is completely replaced if it is no longer present following the administration of the inventive composition. Partial restoration exists where defective tissue remains after the therapeutic composition is administered.

The phrase “effective amount” refers to a concentration or amount of a reagent, pharmaceutical composition, protein, cell population or other agent, that is effective for producing intended result, including treatment of neurodegenerative conditions, or disruption of CD33-CD45 interactions in vitro or in vivo as described herein. An effective amount may vary depending on the specifics of the disorder to be treated, including but not limited to size or total volume/surface area to be treated, as well as proximity of the site of administration to the location of the region to be treated, among other factors familiar to one of skill.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the composition or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

Molecular Engineering

Peptides

In certain aspects, provided herein are peptides that are useful for inhibiting an interaction between a Siglec protein (e.g., CD33) and a ligand of the Siglec protein (e.g., CD45) on a cell surface.

In certain embodiments, provided herein are peptides that comprise an amino acid sequence having at least 83% (e.g., 83%, 84%, 85%, 86%, 87%, 88%, 89%, 90%, 91%, 92%, 93%, 94%, 95%, 96%, 97%, 98%, or 99%) sequence identity to the amino acid sequence of SEQ ID NO: 1 or SEQ ID NO: 3-20, wherein the peptide comprises an arginine, lysine, or phenylalanine residue at the position corresponding to the first or second amino acid of SEQ ID NO: 1 or SEQ ID NO: 3-20. In some embodiments, the peptide comprises an amino acid sequence identical to the amino acid sequence of SEQ ID NO: 1 or SEQ ID NO: 3-20.

In some embodiments, a derivative, equivalent, variant, fragment, or mutant of a peptide described herein or fragment thereof may be suitable for the compositions and methods provided herein. As used herein, the term “conservative substitution” denotes the replacement of an amino acid residue by another, biologically similar residue. It is well known in the art that the amino acids within the same conservative group may typically substitute for one another without substantially affecting the function of a peptide.

Generally, conservative substitutions can be made at any position so long as the required activity is retained. So-called conservative exchanges can be carried out in which the amino acid which is replaced has a similar property as the original amino acid, for example the exchange of Glu by Asp, Gln by Asn, Val by Ile, Leu by Ile, and Ser by Thr. Deletion is the replacement of an amino acid by a direct bond. Positions for deletions include the termini of a peptide or polypeptide and linkages between individual protein domains. Insertions are introductions of amino acids into the polypeptide chain, a direct bond formally being replaced by one or more amino acids. Amino acid sequence can be modulated with the help of art-known computer simulation programs that can produce a peptide or polypeptide with, for example, improved activity or altered regulation. On the basis of this artificially generated peptide or polypeptide sequences, a corresponding nucleic acid molecule coding for such a modulated peptide or polypeptide can be synthesized in-vitro using the specific codon-usage of the desired host cell.

In some embodiments, the peptide is at least 11 amino acids (e.g., at least 11 amino acids, at least 12 amino acids, at least 13 amino acids, etc.) in length. In some embodiments, the peptide is no more than 20 amino acids (e.g., no more than 18 amino acids, no more than 15 amino acids, no more than 12 amino acids, etc.) in length.

In some embodiments, a peptide may be chemically modified. For example, a peptide can be mutated to modify peptide properties such as detectability, stability, biodistribution, pharmacokinetics, half-life, surface charge, hydrophobicity, conjugation sites, pH, function, and the like. N-methylation is one example of methylation that can occur in a peptide of the disclosure. In some embodiments, a peptide may be modified by methylation on free amines such as by reductive methylation with formaldehyde and sodium cyanoborohydride.

A chemical modification may comprise a polymer, a polyether, polyethylene glycol, a biopolymer, a zwitterionic polymer, a polyamino acid, a fatty acid, a dendrimer, an Fc region, a simple saturated carbon chain such as palmitate or myristolate, or albumin. The chemical modification of a peptide with an Fc region may be a fusion Fc-peptide. A polyamino acid may include, for example, a poly amino acid sequence with repeated single amino acids (e.g., poly glycine), and a poly amino acid sequence with mixed poly amino acid sequences that may or may not follow a pattern, or any combination of the foregoing. In some embodiments, the peptides of the present disclosure may be modified such that the modification increases the stability and/or the half-life of the peptides. In some embodiments, the attachment of a hydrophobic moiety, such as to the N-terminus, the C-terminus, or an internal amino acid, can be used to extend half-life of a peptide of the present disclosure. In other embodiments, a peptide may include post-translational modifications (e.g., methylation and/or amidation), which can affect, for example, serum half-life. In some embodiments, simple carbon chains (e.g., by myristoylation and/or palmitylation) can be conjugated to the fusion proteins or peptides. In some embodiments, the simple carbon chains may render the fusion proteins or peptides easily separable from the unconjugated material. For example, methods that may be used to separate the fusion proteins or peptides from the unconjugated material include, but are not limited to, solvent extraction and reverse phase chromatography. The lipophilic moieties can extend half-life through reversible binding to serum albumin. The conjugated moieties may be lipophilic moieties that extend half-life of the peptides through reversible binding to serum albumin. In some embodiments, the lipophilic moiety may be cholesterol or a cholesterol derivative, including cholestenes, cholestanes, cholestadienes and oxysterols. In some embodiments, the peptides may be conjugated to myristic acid (tetradecanoic acid) or a derivative thereof. In other embodiments, a peptide may be coupled (e.g., conjugated) to a half-life modifying agent. Examples of half-life modifying agents include but are not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, a spacer or linker may be coupled to a peptide, such as 1, 2, 3, 4, or more amino acid residues that serve as a spacer or linker in order to facilitate conjugation or fusion to another molecule, as well as to facilitate cleavage of the peptide from such conjugated or fused molecules. In some embodiments, fusion proteins or peptides may be conjugated to other moieties that, for example, can modify or effect changes to the properties of the peptides.

In some embodiments, a chemical modification may include PASylation®, the use of polyglycerols, polyoxazolines, poly(amino acids), polyacylamides, polyvinylpyrrolidones, and polyzwitterons, so long as such changes do not significantly affect the ability of the peptide to provide the therapeutic properties that have been observed.

A peptide may be conjugated to an agent used in imaging, research, therapeutics, theranostics, pharmaceuticals, chemotherapy, chelation therapy, targeted drug delivery, and radiotherapy. In some embodiments, a peptide may be conjugated to or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that can be used in imaging.

In some embodiments, the peptide comprises a sialic-acid binding domain.

In some embodiments, the amino acid at the N-terminus of the peptide is arginine, lysine, or phenylalanine.

In some embodiments, the peptide inhibits an interaction between a Siglec protein (e.g., CD33) and a ligand of the Siglec protein (e.g., CD45).

In certain embodiments, the peptides disclosed herein compete with a peptide of SEQ ID NO: 1 or SEQ ID NO: 3-20 for binding to a ligand of a Siglec protein (e.g. CD33).

In certain embodiments, provided herein is a method of making an inhibitor of an interaction between a Siglec protein and a ligand of the Siglec protein, comprising synthesizing a peptide described herein.

A peptide may be produced recombinantly or synthetically, such as by solid-phase peptide synthesis or solution-phase peptide synthesis. Peptide synthesis may be performed by known synthetic methods, such as using fluorenylmethyloxycarbonyl (Fmoc) chemistry or by butyloxycarbonyl (Boc) chemistry. Peptide fragments may be joined together enzymatically or synthetically.

In certain embodiments, provided herein is a method of inhibiting an interaction between a Siglec protein (e.g., CD33) and a ligand of the Siglec protein (e.g., CD45) on a cell surface (e.g., myeloid cell surface), comprising contacting the ligand of the Siglec protein with a peptide described herein. In some embodiments, inhibiting the interaction between the Siglec protein (e.g., CD33) and the ligand of the Siglec protein (e.g., CD45) decreases an amount of an Amyloid-β peptide (e.g., Amyloid-β peptide 1-42) in the cell (e.g., myeloid cell). In some embodiments, inhibiting the interaction between the Siglec protein (e.g., CD33) and CD45 increases CD45 phosphatase activity.

Exemplary peptides useful for inhibiting an interaction between a Siglec protein (e.g., CD33) and a ligand of the Siglec protein (e.g., CD45) on a cell surface are shown in Table 1 below.

TABLE 1
Exemplary Peptides

| SEQ ID NO. | Sequence |
|---|---|
| SEQ ID NO. 1 | RMESKTEKWMER |
| SEQ ID NO. 3 | RMESKTEKAMER |
| SEQ ID NO. 4 | RMESKTEKFMER |
| SEQ ID NO. 5 | RMESKTEKYMER |
| SEQ ID NO. 6 | RMESQTEKWMER |
| SEQ ID NO. 7 | RMESKTEKWIER |
| SEQ ID NO. 8 | RMESKTEKWMKR |
| SEQ ID NO. 9 | RMESKTEKWMEL |
| SEQ ID NO. 10 | RMESKTEKWMEQ |
| SEQ ID NO. 11 | RFEISEVNRWSD |
| SEQ ID NO. 12 | FRMERGSTKYSY |
| SEQ ID NO. 13 | RGDLGGYNQYTF |
| SEQ ID NO. 14 | FRVERGRDVKYS |
| SEQ ID NO. 15 | FRLKSKWMKYGY |
| SEQ ID NO. 16 | FRMEKGNIKWNY |
| SEQ ID NO. 17 | FRLKRGNMKWSY |
| SEQ ID NO. 18 | FRMEKGSIKWNY |
| SEQ ID NO. 19 | FRVERGSYVRYN |
| SEQ ID NO. 20 | FRVERGSRVRHS |

Nucleic Acids

In certain embodiments, provided herein is a nucleic acid encoding the peptide described herein.

In certain embodiments, provided herein is a vector comprising the nucleic acid described herein.

Useful delivery vectors include but are not limited to biodegradable microcapsules, immuno-stimulating complexes (ISCOMs) or liposomes, and genetically engineered attenuated live carriers such as viruses or bacteria.

In some embodiments, the vector is a viral vector, such as lentiviruses, retroviruses, herpes viruses, adenoviruses, adeno-associated viruses, vaccinia viruses, baculoviruses, Fowl pox, AV-pox, modified vaccinia Ankara (MVA) and other recombinant viruses.

In some embodiments, the nucleic acid or vector comprises an open reading frame encoding a peptide described herein or fragment thereof. In some embodiments, the nucleic acid or vector includes regulatory elements necessary for expression of the open reading frame. Such elements may include, for example, a promoter, an initiation codon, a stop codon, and a polyadenylation signal. In addition, enhancers may be included. These elements may be operably linked to a sequence that encodes a peptide described herein or fragment thereof.

The peptides described herein can be encoded by an expression vector, expression construct, plasmid or recombinant nucleic acid construct.

Examples of promoters include but are not limited to promoters from Simian Virus 40 (SV40), Mouse Mammary Tumor Virus (MMTV) promoter, Human Immunodeficiency Virus (HIV) such as the HIV Long Terminal Repeat (LTR) promoter, Moloney virus, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine, and human metalothionein. Examples of suitable polyadenylation signals include but are not limited to SV40 polyadenylation signals and LTR polyadenylation signals.

In addition, nucleic acids or vectors described herein can include but are not limited to additional regulatory nucleic acid molecules from, e.g., the 3′-untranslated region (3′ UTR). Nucleic acids and vectors described herein can include, but are not limited to, the 5′ untranslated regions (5′ UTR) of an mRNA nucleic acid molecule, which can play an important role in translation initiation and can also be a genetic component in an expression construct. These additional upstream and downstream regulatory nucleic acid molecules may be derived from a source that is native or heterologous with respect to the other elements present on the promoter construct.

In addition to the regulatory elements required for expression, other elements may also be included in the nucleic acid or vector. Such additional elements include enhancers. Enhancers include the promoters described hereinabove. Preferred enhancers/promoters include, for example, human actin, human myosin, human hemoglobin, human muscle creatine and viral enhancers such as those from CMV, RSV and EBV.

Design, generation, and testing of the nucleotides, and their encoded peptides, having a required activity of the expressed protein is within the skill of the art. For example, directed evolution and rapid isolation of mutants can be according to methods described in references including, but not limited to, Link et al., Nature Rev. 5:680-688, 2007; Sanger et al., Gene 97:119-123, 1991; Ghadessy et al., Proc. Natl. Acad. Sci. USA 98:4552-4557, 2001.

In certain embodiments, provided herein is a cell comprising the vector described herein.

Host cells can be transformed using a variety of standard techniques known to the art (see, e.g., Sambrook and Russel, Condensed Protocols from Molecular Cloning: A Laboratory Manual, Cold Spring Harbor Laboratory Press, 2006, ISBN-10: 0879697717; Ausubel et al., Short Protocols in Molecular Biology, 5th ed., Current Protocols, 2002, ISBN-10: 0471250929; Sambrook and Russel, Molecular Cloning: A Laboratory Manual, 3d ed., Cold Spring Harbor Laboratory Press, 2001, ISBN-10: 0879695773; Elhai and Wolk, Meth. Enzymol. 167:747-754, 1988). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier, Protein Expr. Purif. 41:207-234, 2005; Gellissen, ed., Production of Recombinant Proteins: Novel Microbial and Eukaryotic Expression Systems, Wiley-VCH, 2005, ISBN-10: 3527310363; Baneyx, Protein Expression Technologies, Taylor & Francis, 2004, ISBN-10: 0954523253).

Pharmaceutical Compositions & Formulation

In certain aspects, provided herein are pharmaceutic compositions comprising a peptide described herein that are useful for treating or preventing a neurodegenerative disease (e.g., Alzheimer's disease (AD)). In some embodiments, the pharmaceutical composition further comprises a pharmaceutically acceptable excipient.

The pharmaceutical compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a peptide described herein, which can be in purified form, together with a suitable amount of carrier so as to provide the form for proper administration to the subject.

In certain embodiments, the compositions and methods provided herein can be utilized to treat a subject in need thereof. In certain embodiments, the subject is a mammal such as a human, or a non-human mammal. When administered to a subject, such as a human, the composition is preferably administered as a pharmaceutical composition and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In certain embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

In certain embodiments, the pharmaceutical compositions provided herein comprise a pharmaceutically acceptable carrier. A pharmaceutically acceptable carrier can contain physiologically acceptable agents that act, for example, to stabilize, increase solubility or to increase the absorption of a composition. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-microemulsifying drug delivery system. The pharmaceutical composition (preparation) also can be a liposome or other polymer matrix, which can have incorporated therein, for example, a therapeutic compound. Liposomes, for example, which comprise phospholipids or other lipids, are nontoxic, physiologically acceptable and metabolizable carriers that are relatively simple to make and administer.

In certain embodiments, a composition may be simply dissolved or suspended in sterile water.

The formulations may conveniently be presented in unit dosage form and may be prepared by any methods well known in the art of pharmacy. The amount of active ingredient that can be combined with a carrier material to produce a single dosage form will vary depending upon the host being treated, the particular mode of administration. The amount of active ingredient that can be combined with a carrier material to produce a single dosage form will generally be that amount of the composition that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active composition with the carrier and, optionally, one or more accessory ingredients. In general, the formulations are prepared by uniformly and intimately bringing into association a composition with liquid carriers, or finely divided solid carriers, or both, and then, if necessary, shaping the product.

Formulations suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a composition as an active ingredient. Compositions may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered composition moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, oral compositions can also include adjuvants such as wetting agents, emulsifying and suspending agents, sweetening, flavoring, coloring, perfuming, and preservative agents.

Suspensions, in addition to the active compositions, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compositions in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

In certain embodiments, active compositions can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a composition at a particular target site.

Pharmaceutically acceptable salts of composition in the methods provided herein. In certain embodiments, contemplated salts include, but are not limited to, alkyl, dialkyl, trialkyl or tetra-alkyl ammonium salts. In certain embodiments, contemplated salts include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts include, but are not limited to, Na, Ca, K, Mg, Zn, copper, cobalt, cadmium, manganese, or other metal salts.

In certain embodiments, the compositions described herein may comprise wetting agents, emulsifiers and/or lubricants, such as sodium lauryl sulfate and magnesium stearate, as well as coloring agents, release agents, coating agents, sweetening, flavoring and perfuming agents, preservatives and antioxidants.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

In some embodiments, the pharmaceutical composition is formulated to comprise surfactants, buffering agents, and osmolytes to control aggregation.

In some embodiments, the pharmaceutical composition is formulated to comprise enteric-coated capsules, tablets, or beads.

In some embodiments, the pharmaceutical composition is formulated to comprise protease inhibitors to protect against degradation.

In some embodiments, the pharmaceutical composition is formulated to comprise permeation enhancers including EDTA, citric acid, sodium caprate (GIPET®, Merrion Pharmaceuticals), acyl carnitines (Peptelligence®, Enteris), and SNAC (Eligen®, Emisphere), alkylsaccharides, ionic liquids, and nanoparticles.

In some embodiments, the pharmaceutical composition is formulated for extended release.

Controlled-release (or sustained-release) preparations may be formulated to extend the activity of the agent(s) and reduce dosage frequency. Controlled-release preparations can also be used to effect the time of onset of action or other characteristics, such as blood levels of the agent, and consequently affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

In certain embodiments, the pharmaceutical composition formulated for extended release comprises microparticles, liquid crystals, in situ depots, or implants.

Therapeutic Methods

In certain aspects, provided herein are methods of treating or preventing a neurodegenerative disease (e.g., Alzheimer's disease) in a subject comprising administering to the subject a therapeutically effective amount of a pharmaceutical composition described herein.

Exemplary neurodegenerative diseases include, but are not limited to Alzheimer's disease, stroke, Huntington's disease, Parkinson's disease, vascular dementia, senile dementia, frontotemporal dementia, Lewy body dementia, multiple system atrophy, corticobasal degeneration, progressive supranuclear palsy, primary lateral sclerosis, progressive bulbar palsy, progressive muscular atrophy, pseudobulbar pasly, hereditary spastic paraplegia, cerebellar ataxia, amyotrophic lateral sclerosis, HIV associated dementia, cerebral ischemia, amyotrophic lateral sclerosis, multiple sclerosis, Menke's disease, Wilson's disease, Guillain-Barre Syndrome, Creutzfeldt Jakob disease, Fahr disease, prion disease, Huntington's disease, macular degeneration, motor neuron diseases (MND), spinocerebellar ataxia, spinal muscular atrophy, dystonia, idiopathicintracranial hypertension, epilepsy, nervous system disease, central nervous system disease, movement disorders, multiple sclerosis, encephalopathy, peripheral neuropathy, post-operative cognitive dysfunction, cognitive dysfunction arising from viral infection, demyelination in Leukodystrophy, diseases caused by mutation in GALK gene, glioblastoma, and melanoma.

In certain embodiments, the methods may be used to treat Alzheimer's disease (AD). Alzheimer's disease, the most common form of dementia among older adults, is an irreversible degeneration of the brain that causes disruptions in memory, cognition, personality, and other functions that eventually lead to death from complete brain failure.

Genome-wide association studies have identified and validated the innate immune gene CD33 as associated with Alzheimer's disease (AD) susceptibility. CD33 is a sialic acid binding protein expressed on the surface of myeloid cells, and higher CD33 expression levels in the brain have been associated with more advanced cognitive decline and AD. Individuals with the AD associated rs3865444CC risk genotype have increased expression of full-length CD33, the isoform containing the sialic acid binding domain, compared to those with the rs3865444AA protective genotype.

Methods described herein are generally performed on a subject in need thereof. A subject in need of the therapeutic methods described herein can be a subject having, diagnosed with, suspected of having, or at risk for developing a neurodegenerative disease, such as, but not limited to, Alzheimer's disease (AD). A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and chickens, and humans. For example, the subject can be a human subject.

Generally, a safe and effective amount of a pharmaceutical composition described herein is, for example, that amount that would cause the desired therapeutic effect in a subject while minimizing undesired side effects. In various embodiments, an effective amount of peptide pharmaceutical composition described herein can substantially inhibit a neurodegenerative disease, ameliorate a neurodegenerative disease, slow the progress of a neurodegenerative disease, or limit the development of a neurodegenerative disease.

When used in the treatments described herein, a therapeutically effective amount of a pharmaceutical composition described herein can be employed in pure form or, where such forms exist, in pharmaceutically acceptable salt form and with or without a pharmaceutically acceptable excipient. For example, the peptides or pharmaceutical compositions of the present disclosure can be administered, at a reasonable benefit/risk ratio applicable to any medical treatment, in a sufficient amount to treat a neurodegenerative disease, such as Alzheimer's disease.

The amount of a pharmaceutical composition described herein that can be combined with a pharmaceutically acceptable carrier to produce a single dosage form will vary depending upon the host treated and the particular mode of administration. It will be appreciated by those skilled in the art that the unit content of agent, composition or peptide contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD50 (the dose lethal to 50% of the population) and the ED50, (the dose therapeutically effective in 50% of the population). The dose ratio between toxic and therapeutic effects is the therapeutic index that can be expressed as the ratio LD50/ED50, where larger therapeutic indices are generally understood in the art to be optimal.

The specific therapeutically effective dose level for any particular subject will depend upon a variety of factors including the disorder being treated and the severity of the disorder; activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific composition employed; and like factors well known in the medical arts (see, e.g., Koda-Kimble et al., Applied Therapeutics: The Clinical Use of Drugs, Lippincott Williams & Wilkins, 2004, ISBN 0781748453; Winter, Basic Clinical Pharmacokinetics, 4th ed., Lippincott Williams & Wilkins, 2003, ISBN 0781741475; Sharqel, Applied Biopharmaceutics & Pharmacokinetics, McGraw-Hill/Appleton & Lange, 2004, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the peptides and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of an agent, composition or peptide described herein can occur as a single event or over a time course of treatment. For example, an agent, composition or peptide described herein can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

In certain embodiments, pharmaceutical compositions can be used alone or conjointly administered with another type of therapeutic agent (e.g., a cholinesterase inhibitor or memantine). In some embodiments, the additional therapeutic agent is a therapeutic agent for treating Alzheimer's disease. Exemplary Alzheimer's disease therapeutic agents include cholinesterase inhibitors (e.g., (ARICEPT®), rivastigmine (EXELON®)) and galantamine (RAZADYNE®). Cholinesterase and galantamine are inhibitors of the enzyme acetylcholinesterase. Cholinesterase inhibitors work by lowering the brain's normal breakdown of acetylcholine, important in transformation of thought and experience into retrievable memories. The second class of AD drugs enhances the brain's sensitivity to excitatory amino acid neurotransmitter, glutamate and includes memantine (NAMENDA®). The presently described agents, compositions or peptides may be combined with one or more of these agents for administration to a subject. Additional exemplary Alzheimer's disease therapeutic agents include donepezil (Aricept), rivastigmine (Exelon), and suvorextant (Belsomra), and aducanumab.

An agent, composition or peptide described herein can be administered conjointly with another therapeutic agent, such as an antibiotic, an anti-inflammatory, or another agent. For example, an agent, composition or peptide described herein can be administered simultaneously with another agent, such as an antibiotic or an anti-inflammatory. Simultaneous administration can occur through administration of separate compositions, each containing one or more of an agent, composition or peptide described herein, an antibiotic, an anti-inflammatory, or another agent. Simultaneous administration can occur through administration of one composition containing two or more of an agent, composition or peptide described herein, an antibiotic, an anti-inflammatory, or another agent. An agent, composition or peptide described herein can be administered sequentially with an antibiotic, an anti-inflammatory, or another agent. For example, an agent, composition or peptide described herein can be administered before or after administration of an antibiotic, an anti-inflammatory, or another agent.

Administration

In certain aspects, the pharmaceutical compositions provided herein can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); parenterally (including intramuscularly, intravenously, subcutaneously or intrathecally as, for example, a sterile solution or suspension); nasally; subcutaneously; intraperitoneally; and transdermally (for example as a patch applied to the skin). The composition may also be formulated for inhalation. Details of appropriate routes of administration can be found in, for example, U.S. Pat. Nos. 6,110,973; 5,763,493; 5,731,000; 5,541,231; 5,427,798; 5,358,970; and 4,172,896, as well as in patents cited therein.

In certain embodiments, the pharmaceutical compositions provided herein can be administered to a subject by parenteral administration. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compositions in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

In certain embodiments, the pharmaceutical compositions provided herein can be administered to a subject by nanoparticle delivery, viral delivery, or by exosomes.

The pharmaceutical compositions can be used therapeutically either as exogenous agents or as endogenous agents. Exogenous agents are those produced or manufactured outside of the body and administered to the body. Endogenous agents are those produced or manufactured inside the body by some type of device (biologic or other) for delivery within or to other organs in the body.

Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent, composition or peptide in a manner similar to that used for delivering insulin or chemotherapy to specific organs or tumors. Typically, using such a system, an agent, peptide or composition can be administered in combination with a biodegradable, biocompatible polymeric implant that releases the agent, composition or peptide over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents, compositions or peptides can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds., Polymers in Drug Delivery, CRC, 2006, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency, improve taste of the product; or improve shelf life of the product.

In certain embodiments, pharmaceutical compositions can be used alone or conjointly administered with another type of therapeutic agent (e.g., a cholinesterase inhibitor, memantine, donepezil, rivastigmine, suvorextant, or aducanumab).

In certain embodiments, conjoint administration of pharmaceutical compositions with one or more additional therapeutic agent(s) (e.g., a cholinesterase inhibitor or memantine) provides improved efficacy relative to each individual administration of the composition or the one or more additional therapeutic agent(s). In certain such embodiments, the conjoint administration provides an additive effect, wherein an additive effect refers to the sum of each of the effects of individual administration of the therapeutic composition and the one or more additional therapeutic agent(s).

Screening

Also provided are methods for screening. The subject methods find use in the screening of a variety of different candidate molecules (e.g., potentially therapeutic candidate peptides or therapeutic antibodies). Candidate substances for screening according to the methods described herein include, but are not limited to, fractions of tissues or cells, nucleic acids, polypeptides, or peptides.

Kits

Also provided are kits. Such kits can include an agent, composition or peptide described herein and, in certain embodiments, instructions for administration. Such kits can facilitate performance of the methods described herein. When supplied as a kit, the different components of the composition can be packaged in separate containers and admixed immediately before use. Components include, but are not limited to, peptides described herein. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

### EXAMPLES

The following non-limiting example is provided to further illustrate the present disclosure. It should be appreciated by those of skill in the art that the techniques disclosed in the example that follows represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1

Using a crosslinking strategy combined with mass spectrometry, immune cell specific sialic acid-dependent CD33 binding partners were identified, including CD45. These results were validated in vitro as well as in situ. Moreover, it was demonstrated that CD33 modulates the phosphatase activity of CD45 via its sialic acid binding domain.

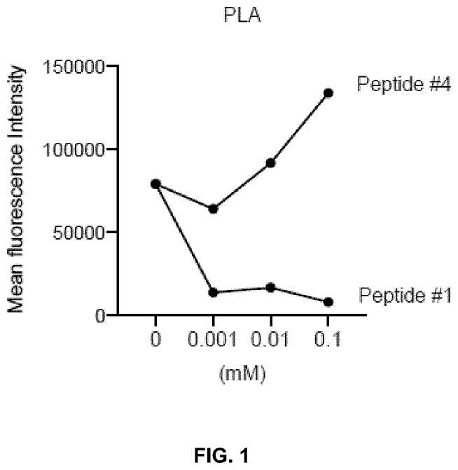

To test if the binding interaction between CD33 and any of its ligands is involved in AD pathogenesis, a 12-mer peptide (RMESKTEKWMER; SEQ ID NO: 1) was designed based on the structure of the sialic-acid binding domain of siglecs (Sialic acid-binding immunoglobulin-type lectins). First, it was demonstrated that this inhibitory peptide (SEQ ID NO: 1) disrupts the interaction between CD33 and CD45 (FIG. 1). SEQ ID NO: 1 (Peptide #1; 0-0.1 mM) reduces the interaction between CD33 and CD45 in the human myeloid cell line THP1 using the proximity ligation assay. This is not true of a peptide (MESKTEKWMERI; SEQ ID NO: 2; peptide #4), which does not contain the initial arginine amino acid residue.

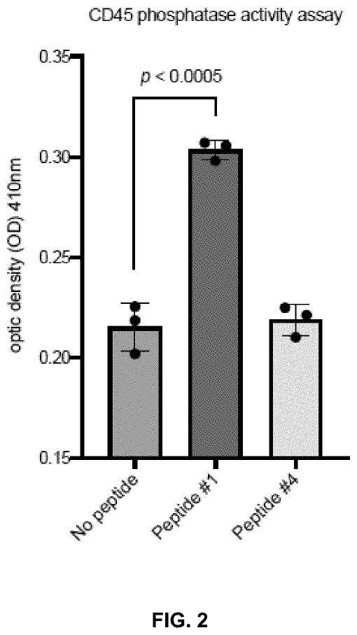

Second, it was found that SEQ ID NO: 1, which disrupts the CD33-CD45 protein interaction, increases CD45 phosphatase activity in vitro consistent with CD33 being a negative regulator of CD45 function (FIG. 2). The addition of SEQ ID NO:1 (peptide #1) to THP1 cells leads to an increase in CD45 phosphatase activity, while addition of SEQ ID NO:2 (peptide #4) lacking the initial arginine residue, does not.

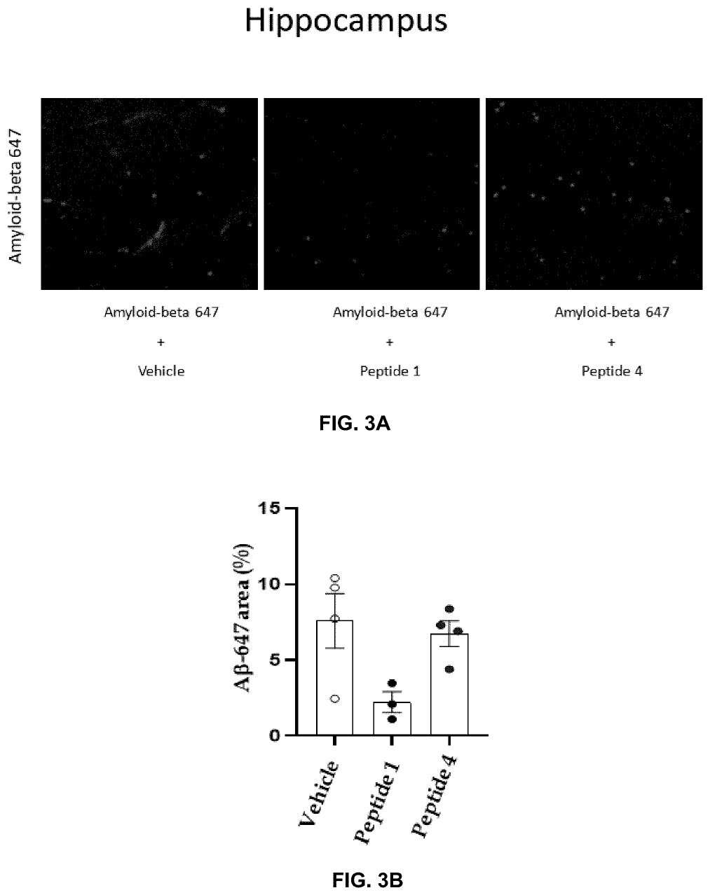

Third, by leveraging an in vivo model of microglia-mediated amyloid uptake in the mouse brain, and using osmotic mini pumps implanted subcutaneously to deliver the peptide through a catheter connected to the brain ventricles, it was shown that the inhibitory peptide SEQ ID NO:1 (peptide #1) reduces the amount of exogenous amyloid-beta 1-42 detectable in the brains of animals (FIG. 3A and FIG. 3B). 10 uM peptide solutions, containing SEQ ID NO: 1 (peptide #1) or SEQ ID NO: 2 (peptide #4), were added to osmotic mini pumps to permit daily delivery of approximately 5 ul of dosing solution or 3 ug/kg of peptide. A fluorescently labelled analog of amyloid beta 1-42 (Amyloid-beta 647) was delivered via ICV infusion. Confocal microscopy was later used to quantify signal in hippocampal sections. The addition of SEQ ID NO: 1 (Pept1) but not control SEQ ID NO: 2 (Pept4) decreases the amount of exogenous Amyloid-beta 1-42 in vivo. Both the fluorescent labelled amyloid-beta (red) and peptides were directly administered to the brain via a mini osmotic pump implanted subcutaneously and connected to the brain ventricles through a catheter for continuous drug delivery. By the end of the treatment (28 days), brain sections were performed and analyzed by confocal microscope.

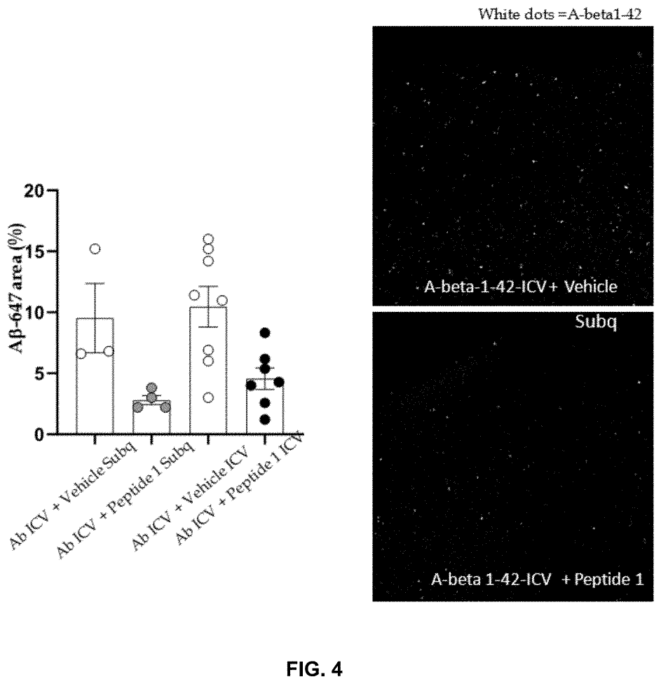

Hippocampus areas are shown of the three treated mice (FIG. 3A). Quantification of Amyloid-beta 1-42 in the hippocampus from the three mouse groups are shown with a marked decrease of Amyloid-beta 1-42 in mice that received Peptide #1 but not those that were treated with Peptide #4 (FIG. 3B). SEQ ID NO: 1 (Pept1) also reduced levels of amyloid-beta 1-42 in brain when osmotic mini pumps were used to deliver the peptide to the periphery (FIG. 4). The addition of SEQ ID NO: 1 (Pept1) decreases the amount of exogenous Amyloid-beta 1-42 in brain when the peptide is delivered subcutaneously (subq). The fluorescent labelled A-beta (red) was directly administered to the brain via a mini osmotic pump implanted subcutaneously and connected to the brain ventricles through a catheter for continuous drug delivery. The peptide (SEQ ID NO: 1) was delivered subcutaneously via another pump. By the end of the treatment (28 days), brain sections were analyzed by confocal microscope. Quantification of Amyloid-beta 1-42 in the parenchyma from each mouse group are shown with a marked decrease of Amyloid-beta 1-42 in mice that received SEQ ID NO: 1 compared to those that received SEQ ID NO: 4 and mice that did not receive any peptide.

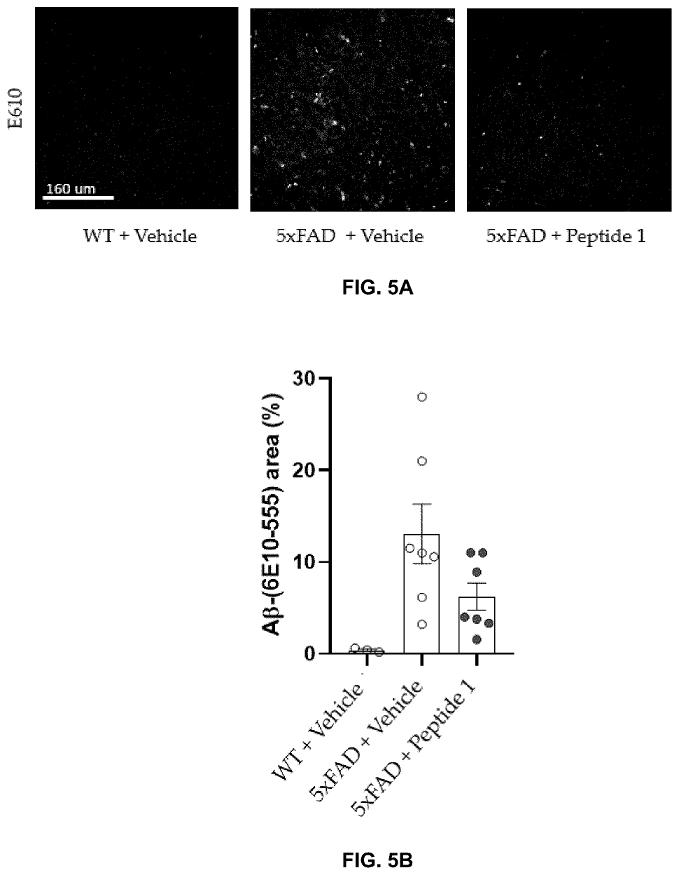

Fourth, by leveraging a second in vivo model of microglia-mediated amyloid uptake in the mouse brain, and using osmotic mini pumps implanted subcutaneously to deliver the peptide through a catheter connected to the brain ventricles, it was shown that the inhibitory peptide SEQ ID NO:1 (peptide #1) reduces the amount of endogenous amyloid-beta 1-42 detectable in the brains of 5×FAD animals (FIG. 5A and FIG. 5B). 5×FAD mice express human APP and PSEN1 transgenes that include five AD-linked mutations. These mice recapitulate many AD-related phenotypes and have a relatively early and aggressive presentation. Amyloid plaques, accompanied by gliosis, are seen in mice as young as two months of age. Neuron loss occurs in multiple brain regions, beginning at about 6 months in the areas with the most pronounced amyloidosis. Mice display a range of cognitive and motor deficits.

The addition of SEQ ID NO: 1 (Pept1) but not vehicle control decreases the amount of endogenous Amyloid-beta 1-42 in vivo. Peptide was directly administered to the brain via a mini osmotic pump implanted subcutaneously and connected to the brain ventricles through a catheter for continuous drug delivery. By the end of the treatment (28 days), brain sections were generated and analyzed by confocal microscope. A representative image from an animal in each of the three groups is shown (FIG. 5A). Quantification of E610 immunoreactivity in the hippocampus, reflective Amyloid-beta 1-42 levels, from the three mouse groups is shown. A marked decrease of Amyloid-beta 1-42 was observed in 5×FAD mice that received Peptide #1 but not those that received vehicle (FIG. 5B).

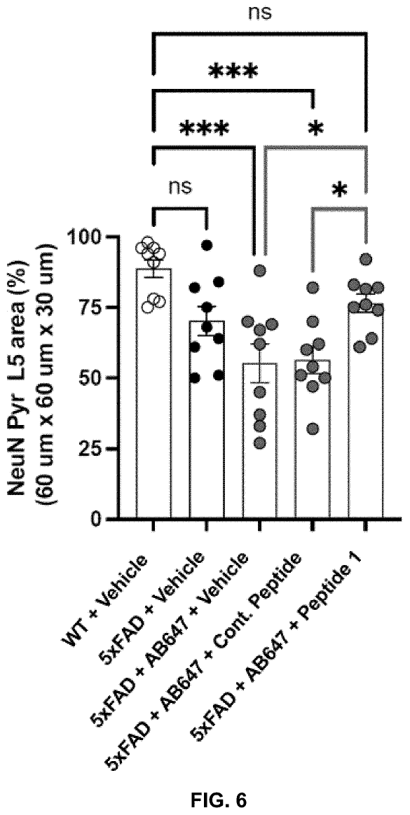

Fifth, in a modified version of this second in vivo model of Alzheimer's Disease, and using osmotic mini pumps implanted subcutaneously to deliver the peptide through a catheter connected to the brain ventricles, it was shown that the inhibitory peptide SEQ ID NO:1 (peptide #1) reduces neuronal loss in transgenic 5×FAD animals treated with exogenous Amyloid-beta 1-42 (FIG. 6). The addition of SEQ ID NO: 1 (Pept1) but not vehicle or a control peptide (KMRWETMESREK; SEQ ID NO: 21) increased neuronal survival in vivo. Peptide was directly administered to the brain via a mini osmotic pump implanted subcutaneously and connected to the brain ventricles through a catheter for continuous drug delivery. By the end of the treatment (28 days), brain sections were generated and analyzed by confocal microscope. Quantification of NeuN immunoreactivity in Layer 5 of the piriform cortex, reflective of neuronal number, from the three mouse groups is shown. FIG. 6 shows administration of SEQ ID NO: 1 (peptide #1) increased neuronal survival in 5×FAD transgenic mice administered exogenous amyloid-beta 1-42 in comparison to 5×FAD/exogenous amyloid-beta 1-42 animals treated with vehicle or a control peptide (Scrambled).

Altogether, the present study identified a novel biologic (SEQ ID NO: 1) that disrupts the interaction between an AD risk molecule (CD33) and a signaling protein (CD45) expressed on myeloid cells. Therefore targeting this protein-protein interaction using a small peptide has protective effects in reducing the amount of toxic amyloid-beta 1-42.

### INCORPORATION BY REFERENCE

All publications, patents, patent applications, and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

1. A peptide of no more than 30 amino acids in length and comprising an amino acid sequence having at least 83% sequence identity to the amino acid sequence of SEQ ID NO: 1, wherein the peptide comprises an arginine, lysine, or phenylalanine residue at the position corresponding to the first or second amino acid of SEQ ID NO: 1 or SEQ ID NO: 3-20.

2. The peptide of claim 1, wherein:
a) the peptide comprises an amino acid sequence having at least 91% sequence identity to the amino acid sequence of SEQ ID NO: 1 or SEQ ID NO: 3-20; or
b) the peptide comprises an amino acid sequence identical to the amino acid sequence of SEQ ID NO: 1 or SEQ ID NO: 3-20.

3. The peptide of claim 1, wherein:
a) the peptide is at least 11 amino acids in length;
b) the peptide is at least 12 amino acids in length;
c) the peptide is no more than 20 amino acids in length;
d) the peptide is no more than 15 amino acids in length; or
e) the peptide is no more than 12 amino acids in length.

4. The peptide of claim 1, wherein the peptide comprises a chemical modification.

5. The peptide of claim 4, wherein:
a) the peptide is chemically modified with polyethylene glycol (PEG), a glycan, an acetic acid, an amide, a fatty acid, a phosphoryl group, a methyl group, or a combination thereof;
b) the peptide is chemically modified with small residues Pro, Ala and Ser (PAS);
c) the peptide is chemically modified with polyglycerol, polyoxazoline, polyamino acid, polyacylamide, polyvinylpyrrolidone, zwitterionic polymer, biopolymer, dendrimer, polyether, or polyethylene glycol, or a combination thereof;
d) the peptide is chemically modified with cholesterol, cholestene, cholestane, cholestadiene, oxysterol, or a combination thereof; or
e) the peptide is chemically modified with palmitate, myristolate, albumin, or a combination thereof.

6. The peptide of claim 1, wherein:
a) the peptide comprises a sialic-acid binding domain;
b) the amino acid at the N-terminus of the peptide is arginine, lysine, or phenylalanine; or
c) the peptide inhibits an interaction between a Siglec protein and a ligand of the Siglec protein.

7. The peptide of claim 6, wherein the Siglec protein is CD33.

8. The peptide of claim 7, wherein the ligand of the Siglec protein is CD45.

9. The peptide of claim 1, wherein the peptide competes with a peptide of SEQ ID NO: 1 or SEQ ID NO: 3-20 for binding to a ligand of a Siglec protein.

10. The peptide of claim 9, wherein the Siglec protein is CD33.

11. The peptide of claim 10, wherein the ligand of the Siglec protein is CD45.

12. A pharmaceutical composition comprising the peptide of claim 1.

13. The pharmaceutical composition claim 12, further comprising a pharmaceutically acceptable excipient.

14. The pharmaceutical composition of claim 12, wherein the composition is formulated for extended release.

15. A method of inhibiting an interaction between a Siglec protein and a ligand of the Siglec protein on a cell surface, comprising contacting the Siglec protein and/or the ligand of the Siglec protein with the peptide of claim 1.

16. The method of claim 15, wherein:
a) inhibiting the interaction between the Siglec protein and the ligand of the Siglec protein decreases an amount of an Amyloid-β peptide in the cell;
b) the ligand of the Siglec protein is CD45;
c) the Siglec protein is CD33; or
d) the cell is a myeloid cell.

17. The method of claim 16, wherein:
a) the Amyloid-β peptide is Amyloid-β peptide 1-42; or
b) inhibiting the interaction between the Siglec protein and CD45 increases CD45 phosphatase activity.

18. A method of treating or preventing a neurodegenerative disease in a subject comprising administering to the subject a therapeutically effective amount of the pharmaceutical composition of claim 12.

19. The method of claim 18, wherein:
a) the neurodegenerative disease is Alzheimer's disease;
b) the administration is subcutaneous injection, intravenous injection, intramuscular injection, intrathecal injection, microneedle injection, intravenous infusion, oral administration, sublingual administration, or intranasal administration;
c) the administration decreases the amount of Amyloid-β peptide in the nervous system of the subject;
d) the Amyloid-β peptide is a parenchymal Amyloid-β peptide;
e) the Amyloid-β peptide is exogenous Amyloid-β peptide;
f) the Amyloid-β peptide is endogenous Amyloid-β peptide;
g) the Amyloid-β peptide is a pathogenic Amyloid-β peptide; or
h) the Amyloid-β peptide is Amyloid-β peptide 1-42.

20. The method of claim 18, further comprising conjointly administering an additional therapeutic to the subject.

21. The method of claim 20, wherein the additional therapeutic is a cholinesterase inhibitor, memantine, donepezil, rivastigmine, suvorextant, or aducanumab.

22. A method of making an inhibitor of an interaction between a Siglec protein and a ligand of the Siglec protein, comprising synthesizing the peptide of claim 1.

23. A nucleic acid encoding the peptide of claim 1.

24. A vector comprising the nucleic acid of claim 23.

25. A cell comprising the vector of claim 24.


SEQUENCE LISTING (20 sequences)

SEQ ID NO: 1 - protein, 12 aa, synthetic construct
  REGION 1..12: note = Synthetic
RMESKTEKWMER

SEQ ID NO: 2 - protein, 12 aa, synthetic construct
  REGION 1..12: note = Synthetic
MESKTEKWMERI

SEQ ID NO: 3 - protein, 12 aa, synthetic construct
  REGION 1..12: note = Synthetic
RMESKTEKAMER

SEQ ID NO: 4 - protein, 12 aa, synthetic construct
  REGION 1..12: note = Synthetic
RMESKTEKFMER

SEQ ID NO: 5 - protein, 12 aa, synthetic construct
  REGION 1..12: note = Synthetic
RMESKTEKYMER

SEQ ID NO: 6 - protein, 12 aa, synthetic construct
  REGION 1..12: note = Synthetic
RMESQTEKWMER

SEQ ID NO: 7 - protein, 12 aa, synthetic construct
  REGION 1..12: note = Synthetic
RMESKTEKWIER

SEQ ID NO: 8 - protein, 12 aa, synthetic construct
  REGION 1..12: note = Synthetic
RMESKTEKWMKR

SEQ ID NO: 9 - protein, 12 aa, synthetic construct
  REGION 1..12: note = Synthetic
RMESKTEKWMEL

SEQ ID NO: 10 - protein, 12 aa, synthetic construct
  REGION 1..12: note = Synthetic
RMESKTEKWMEQ

SEQ ID NO: 11 - protein, 12 aa, synthetic construct
  REGION 1..12: note = Synthetic
RFEISEVNRWSD

SEQ ID NO: 12 - protein, 12 aa, synthetic construct
  REGION 1..12: note = Synthetic
FRMERGSTKYSY

SEQ ID NO: 13 - protein, 12 aa, synthetic construct
  REGION 1..12: note = Synthetic
RGDLGGYNQYTF

SEQ ID NO: 14 - protein, 12 aa, synthetic construct
  REGION 1..12: note = Synthetic
FRVERGRDVKYS

SEQ ID NO: 15 - protein, 12 aa, synthetic construct
  REGION 1..12: note = Synthetic
FRLKSKWMKYGY

SEQ ID NO: 16 - protein, 12 aa, synthetic construct
  REGION 1..12: note = Synthetic
FRMEKGNIKWNY

SEQ ID NO: 17 - protein, 12 aa, synthetic construct
  REGION 1..12: note = Synthetic
FRLKRGNMKWSY

SEQ ID NO: 18 - protein, 12 aa, synthetic construct
  REGION 1..12: note = Synthetic
FRMEKGSIKWNY

SEQ ID NO: 19 - protein, 12 aa, synthetic construct
  REGION 1..12: note = Synthetic
FRVERGSYVRYN

SEQ ID NO: 20 - protein, 12 aa, synthetic construct
  REGION 1..12: note = Synthetic
FRVERGSRVRHS